SEQUENCE LISTING (2 sequences)

SEQ ID NO: 1 - protein, 29 aa, Rabies virus
YTIWMPENPRPGTPCDIFTNSRGKRASNG

SEQ ID NO: 2 - protein, 40 aa, Homo sapiens
CGRLVQYRGEVQAMLGQSTEELRVRLASHLRKLRKRLLRD

(19) United States
(12) Patent Application Publication
(10) Pub. No.: US 2021/0196750 A1
(43) Pub. Date: Jul. 1, 2021

(54) Liposomal-gold nanoparticles for drug delivery into the brain

(71) Applicants: Pablo Vivas-Mejia, San Juan, PR (US); Nilmary Grafals, San Juan, PR (US)

(72) Inventors: Pablo Vivas-Mejia, San Juan, PR (US); Nilmary Grafals, San Juan, PR (US)

(21) Appl. No.: 17/063,423

(22) Filed: Oct. 5, 2020

Related U.S. Application Data
(60) Provisional application No. 62/910,287, filed on Oct. 3, 2019.

(51) Int. Cl. A61K 33/242; A61K 9/127; C12N 15/113; A61K 47/69; A61K 47/62; A61P 35/00

(52) U.S. Cl. CPC A61K 33/242; A61K 9/1275; C12N 15/113; C12N 2320/32; A61K 47/62; A61P 35/00; C12N 2320/31; A61K 47/6911

(57) ABSTRACT

The present invention discloses targeting microRNAs as a potential therapeutic target for cancer, more particularly glioblastoma (GBM). It discloses targeting microRNAs with gold-nanoliposomes labeled with brain targeting-peptides inducing a significant cell growth arrest and inhibition of miRNA-92b, an aberrantly abundant miRNA found in GBM cells. Furthermore, it delivers gold-nanoliposomes to the brain by crossing the blood brain barrier (BBB) and reaching cancer tumors.

### RELATED APPLICATIONS

The present application claims priority to Provisional U.S. application Ser. No. 62/910,287, filed on Oct. 3, 2019, all of which is hereby incorporated by reference in its entirety.

### STATEMENT REGARDING FEDERALLY SPONSORED RESEARCH AND DEVELOPMENT

This project is supported in part by the National Institutes of Health (NIH)-U54 MD007600 and the Minority Biomedical Research Support (MBRS) RISE Grant Number R25-GM061838.

### BACKGROUND OF THE DISCLOSURE

### Discussion of the Background

Glioblastoma (GBM) is the most common and malignant of all primary brain tumors, being responsible for over 14,000 annual deaths in the U.S. (National Cancer Institute). The most common treatment for GBM patients consists of tumor resection in combination with radiotherapy and/or temozolomide (TMZ) chemotherapy. With this standard of care, GBM patients usually survive no more than 2 years after initial diagnosis. Other treatment alternatives include stereotactic radiotherapies (SRS) for localized tumor radiation, antibodies against the Vascular Endothelial Factor using bevacizumab (FDA approved for recurrent GBM); and localized administration of chemotherapies, also known as convection enhanced delivery (CED). Despite the arsenal of diagnosis tools and targeted therapies, the overall survival rate of GBM patients has barely improved over the last 20 years. Therefore, novel and more effective therapies are urgently needed.

Recently, there has been an increased interest in the RNA interference (RNAi) as a therapeutic modality for cancer therapy. Two RNAi-based therapies are currently investigated. The first one, called small-interference RNA (siRNA), uses a double stranded RNA oligonucleotide (21-27 base-pairs) to target the messenger RNA (mRNA) of a specific gene that is unregulated in cancer cells. The second RNAi-based therapy is designed to target miRNAs deregulated in cancer cells with RNA-OLN. MiRNAs are endogenous small non-coding RNAs (22 nucleotides in length) that regulate gene expression at the post-transcriptional level. Evidence indicates that miRNA dysregulation contributes to GBMs' initiation, progression, and infiltration ability. While some of these miRNAs are downregulated and act as tumor suppressor genes, others are upregulated and may acts as oncogenes. MiRNA-based therapies with RNA-OLN are designed to target upregulated miRNAs with oligonucleotide miRNA inhibitors (OMIs) or against downregulated miRNA with oligonucleotide miRNA mimics (OMMs).

Despite promising outcomes in the bench, translation to the clinic of RNAi-based therapies has been halted for reasons that include their fast renal clearance, their propensity for nuclease degradation, their low incorporation into cancer cells, and the activation of immune responses.

Nanoparticles with different materials and physicochemical properties have been designed to improve delivery efficiency of RNAi, and for many other commonly used anticancer agents. They can protect RNAi from degradation and enhance their circulatory half-lives. Nanoparticles with sizes of 50-200 nm can also accumulate in tumor tissues due to imperfect vascularization—a trademark known as the enhanced permeability retention effect (EPR). Moreover, most nanoparticles can be functionalized (labeled) with oligonucleotides, proteins and peptides to increase their specificity for the site of interest. Liposomes are the most studied delivery systems for cancer therapeutic as they can be prepared with biodegradable and biocompatible materials. More recently, gold nanoparticles (AuNP) have also been proposed for drug delivery which are of easily preparation and conjugation. Because of their high surface area and spherical shape, conjugation of oligonucleotides to AuNP core leads to a 3D structure commonly known as Spherical Nucleic Acids (SNAs). An additional challenge regarding GBM treatment with RNAi (and with all drugs in general) is the presence of the BBB. The BBB is an anatomical semipermeable barrier that protects the brain from alien substances and regulate the movement of specific molecules from blood to the brain (and vice versa). In fact, more than 98% of potential drugs for brain-related diseases fail to cross the BBB. Therefore, creation of nanoparticles for efficient delivery of RNAi-based therapies capable to cross the BBB is of great clinical advancement. A common mechanism where molecules in circulation interact with receptors of brain capillary endothelial cells and cross the BBB is the receptor mediated transcytosis. This mechanism of transport has been thoroughly investigated as a promising pathway to transport nanoparticles through the BBB into brain tumor cells. Apolipoprotein E (ApoE) and Rabies Virus Glycoprotein (RVG) have proven to effectively transport biomolecules to the brain through receptor mediated transcytosis. ApoE is specific for LDL Receptors, highly expressed in brain vascular endothelial cells and in GBM cells. Annika Böckenhoff, et. al. showed that ApoE was the most effective peptide to deliver the lysosomal enzyme arylsulfatase A (ASA) to the brain of wild type mice, in comparison to other brain specific peptides (TAT, Angiopep peptide and Apolipoprotein B). RVG on the other hand, is specific for the nicotinic Acetylcholine receptors, highly expressed in brain endothelium, neurons and in GBM cells. Priti Kumar, et. al. showed that conjugating siRNAs to RVG peptides could increase their delivery into the brain rather than liver or spleen of wild type mice.

It is, therefore, desirable to have a method and compound for the treatment of cancer that incorporates into cancer cells. It is also desirable to have the creation of nanoparticles for efficient delivery of RNAi-based therapies which are capable to cross the BBB. Therefore, the present disclosure is directed to overcome the problems or disadvantages associated with the prior art.

### SUMMARY OF THE DISCLOSURE

The present disclosure is related to synthesized nanoparticles for delivering OMIs inside GBM cells. In accordance with the present disclosure, SNAs are produced by chemically conjugating OMIs to AuNPs. Then, SNAs are encapsulated inside ApoE or RVG peptides-conjugated liposomes. Therefore, OMIs-containing liposomes labeled with ApoE or RVG peptides were prepared.

Another object of the present disclosure is to decrease the expression of miR-92b in a GBM cell line. In accordance with the present disclosure, the synthesized nanoparticles decrease the expression of miR-92b in a GBM cell line, which is an aberrantly abundant miRNA in GBM cells and GBM patient samples.

According to another aspect, the present disclosure serves to provide a nanocarrier to accumulate into brain tumor tissue of syngeneic GBM bearing mammal after vein injection. In accordance with the principal of the present invention the SNALiposomes that were labeled with RVG or ApoE significantly increased the accumulation into GBM tumor tissue. Further, SNA-Liposome-ApoE showed to be the most effective RNAi delivering system in vitro and in vivo.

According to another aspect, the present disclosure serves to provide a dual nanoparticle capable of RNAi delivery to GBM cells.

According to another aspect, the present disclosure is directed to a method for the treatment of cancer, more particularly GBM, by synthesizing nanoparticles for delivering RNAi-based therapies to cross the BBB.

According to another aspect, the present disclosure serves to provide a synthesized SNA-Liposome that reduces GBM cell viability and increased nanoparticle accumulation into intracranial GBM mouse tumors.

The present disclosure may address one or more problems and deficiencies of the prior art discussed above. However, it is contemplated that the disclosure may prove useful in addressing other problems and deficiencies in a number of technical areas. Therefore, the claimed invention should not necessarily be construed as limited to addressing any of the particular problems or deficiencies discussed herein.

### BRIEF DESCRIPTION OF THE DRAWINGS

The accompanying drawings, which are incorporated herein, constitute part of the specification and illustrate the preferred embodiment of the disclosure.

FIG. 1. Physicochemical Characteristics of SNAs

FIG. 2. Physicochemical Characteristics of OMIs-Containing Liposomes and SNA-Containing Liposomes

FIG. 3. Preparation of SNAs and peptide labeled liposomes.

FIG. 4. Characterization of SNAs and in vitro toxicity.

FIG. 5. Physical characterization of nanoparticles.

FIG. 6. Cell internalization measurements.

FIG. 7. Confirmation of microRNA inhibition in U87 cells.

FIG. 8. Fluorescence microscopy images showing the nanoparticle accumulation in brain tumor-adjacent areas of GBM syngeneic mice.

FIG. 9. Fluorescence microscopy images showing endosomal escape.

FIG. 10. DLS analysis for AuNPs-Liposome-ApoE

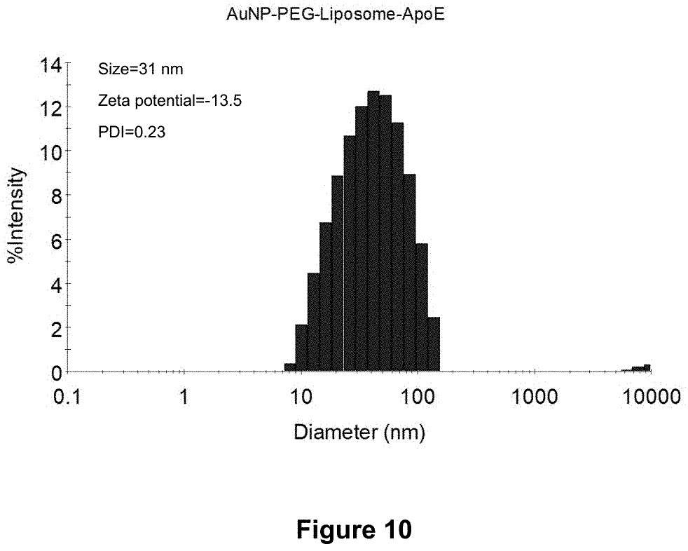

FIG. 11. Fluorescent intensity analysis for U87 nanoparticle uptake.

FIG. 12. Real-time PCR-based miR-92b expression in U87 cells after treatment with nanoparticles containing NC-OMIs and miR92b-OMIs

FIG. 13. Histological confirmation of GBM tumor growth in the brain of syngeneic mice

FIG. 14. Fluorescence microscopy image analysis of nanoparticle accumulation in brain tumors of GBM syngeneic mice:

FIG. 15. Fluorescence microscopy images of nanoparticle accumulation in liver tissues of GBM syngeneic mice.

FIG. 16. Cell viability of U87 cells incubated with miR-92b-targeted SNA-Liposome-ApoE.

FIG. 17. RNA stability of naked OMIs and SNA-Liposome-ApoE after incubation in 30% FBS at 37° C.

FIG. 18. Shelf-life of SNA-Liposome-ApoE nanoparticles.

### DETAILED DESCRIPTION

Detailed descriptions of one or more preferred embodiments are provided herein. It is to be understood, however, that the present disclosure may be embodied in various forms. Therefore, specific details disclosed herein are not to be interpreted as limiting, but rather as a basis for the claims and as a representative basis for teaching one skilled in the art to employ the present invention in any appropriate system, structure or manner.

The present disclosure relates to deregulation of microRNAs (miRNA), a common phenomenon observed in many cancer cells, and is associated with cancer initiation, progression, tumor maintenance, and drug resistance. Targeting microRNAs (miRNA) has been proposed as a novel therapy for cancer treatment, including glioblastoma (GBM), the most common type of the Central Nervous System (CNS) malignancy. Limitations of miRNA-based therapies with RNA oligonucleotides (RNA-OLN) include their poor stability, off-target effects, and activation of immune responses. Part of these challenges have been solved with the creation of nanoparticles which protect RNA-OLN from degradation in the circulation. For the treatment of GBMs, RNA-OLN-based therapies confront an additional obstacle as they need to cross the blood brain barrier (BBB), which precludes 98% of potential drugs from reaching the brain. Aiming to develop oligonucleotide nanocarriers that can potentially cross the BBB and reach GBM tumors, we created novel gold-nanoliposomes labeled with brain targeting-peptides, Apolipoprotein E (ApoE) and Rabies Virus Glycoprotein (RVG). Here, we functionalized 15 nm gold nanoparticles (AuNP) to thiol-containing oligonucleotide miRNA inhibitors (OMIs), a type of RNA-OLN used against aberrantly abundant miRNAs. The AuNP-OMI nanoparticle, commonly called spherical nucleic acids (SNAs), are nontoxic in GBM cell lines and have a loading capacity of 50 moles of OMI per mole of AuNP. After encapsulating SNAs into ApoE and RVG peptide-labeled liposomes, we obtained 30-50 nm nanoparticles (called SNA-Liposome-ApoE and SNA-Liposome-RVG) with slight negative charge and with encapsulation efficiencies greater than 70%. In vitro studies in GBM cells showed that SNA-Liposomes were highly effective at internalizing cells and inhibiting the expression of miRNA-92b, an aberrantly abundant miRNA in GBM cells. In addition, intravenous administration of SNA-Liposomes labeled with ApoE or RVG peptides were accumulated in the brain tumor tissue of a GBM syngeneic mouse model. This study is an additional step for the translation of miRNA-based therapies not only for GBM, but also for other neurodegenerative disorders.

Materials and Methods

Chemicals and Reagents

AuNPs, Dubelcco's Phosphate Buffer Saline (PBS), DL-Dithiothreitol (DTT), Tert-butanol and 50 KDa micro-dialysis membranes were purchased from Sigma-Aldrich (St. Louis, Mo., USA). Nap-10 G-25 Sephadex Columns were obtained from GE Lifesciences (Pittsburgh, Pa., USA). DSPE-PEG-2000 (1,2-distearoyl-sn-glycero-3-phosphoethanolamine-N-[methoxy(polyethylene glycol)-2000]), DOPC (1,2-dioleoyl-sn-glycero-3-phophocholide), and cholesterol were purchased from Avanti Polar Lipids (Alabaster, Ala., USA). DSPE-PEG-Maleimide and mPEG-SH (SH=thiol or sulfhydryl group) were purchased from Nanocs (New York, N.Y., USA). We obtained RVG (YTIWMPENPRPGTPCDIFTNSRGKRASNG) and ApoE (CGRLVQYRGEVQAMLGQSTEELRVRLASHLRKLRKRLLRD) from Anaspec (Freemont, Calif., USA) and Lifetein (Somerset, N.J., USA), respectively. MirVana miRNA inhibitors (OMIs) were obtained from Thermo Fisher Scientific (Austin, Tex., USA) which includes negative Control #1, SH-Negative Control #1 (5′-sequence-SH-3′), antimiR-92b, SH-antimiR-92b, Negative Control #1-Alexa Fluor 647, and SH-negative control #1-Alexa Fluor 647 (5′-Alexa Fluor 647-sequence-SH-3′). Mercaptoethanol was purchased from Bio-Rad (Berkeley, Calif., USA). The “measure IT Thiol Assay Kit” was purchased from Thermo Fisher Scientific (Waltham, Mass., USA) and the “maleimide Quantification Assay Kit” was purchased from Abcam (Cambridge, UK).

Cell Line and Culture Conditions

The U87 GBM cell line was purchased from the American Type Culture Collection (Manassas, Va., USA). GL261 cells (astrocytomas from mouse) were a kind gift from Dr. Lilia Y Kucheryavykh. Both cells were grown as adherent cells and maintained in DME/F12 Media from HyClone Lab (Logan, Utah, USA) supplemented with 10% of fetal bovine serum (FBS) (Invitrogen) and 0.1% of penicillin/streptomycin (Invitrogen) at 37° C. in a humid atmosphere with 5% CO2 (normal cell conditions). Cells were used for in vitro experiments at a confluence of 75-85%.

Spherical Nucleic Acid (SNA) Synthesis

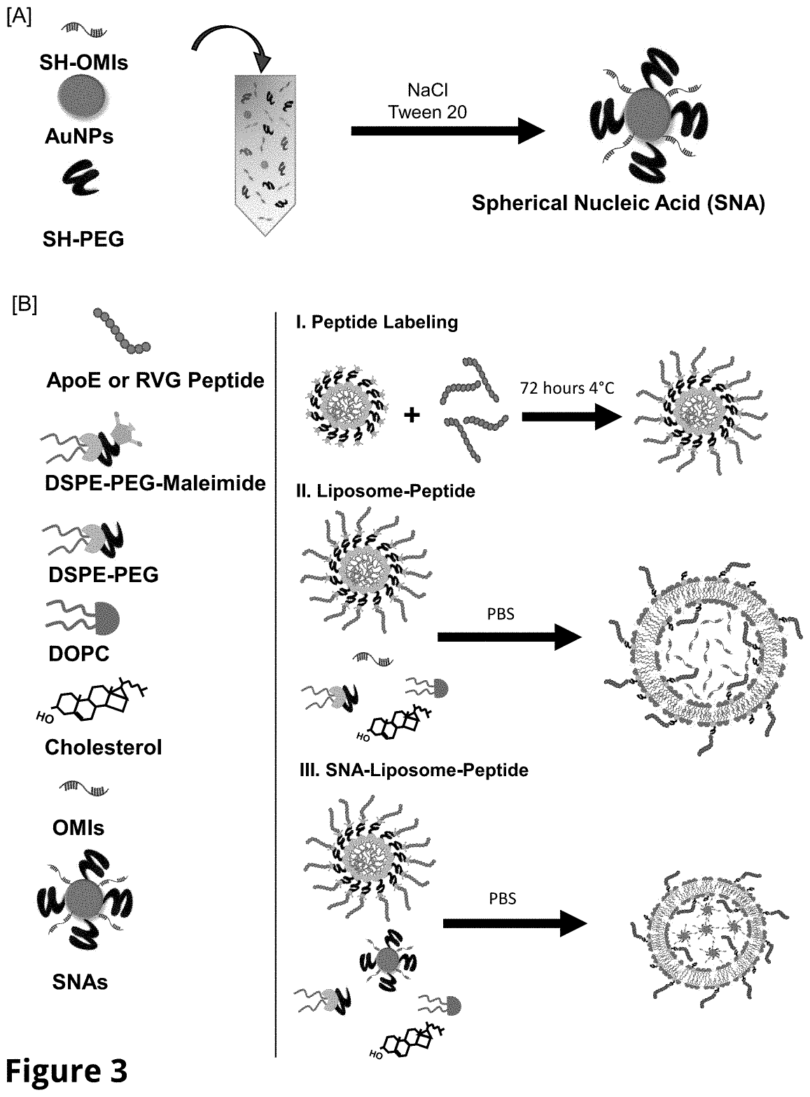

To synthesize spherical nucleic acids (SNAs) we used the PEG and Tween-20 stabilization methods described by Jiuxing Li, et. al 37, with some modifications (FIG. 3, section A). First, SH-OMIs were reduced with 50 mM of DTT during 1.5 hours at room temperature and purified with Nap-10 G25 Sephadex columns prior to conjugation reaction. Citrate capped 15 nm AuNPs (Sigma) resuspended in deionized water (ddH2O) were mixed with mPEG(2000)-SH and Tween-20 for 30 minutes at room temperature. Then, filtered SH-OMIs were added to the mixture and vortexed. The final concentrations of each of the four components were as follow: 1.5 nM for AuNPs, 100 nM for mPEG-SH, 1 mM for Tween-20, and 450 nM for SH-OMIs. NaCl was added to a final concentration of 1M and incubated for 1.5 hours while rotating at room temperature. Salt and reagent excess were removed by two consequent centrifugations of 17,000 RCF at 4° C. for 30 minutes and resuspended with PBS 1×. The SH-OMIs conjugated to AuNPs are the spherical nucleic acids (SNAs). SNAs concentration was calculated by using the Beer Lambert Equation, using AuNP's absorbance of 520 nm UV-visible and an extinction coefficient of 3.67×108 M-1 cm-1.

SNAs Oligonucleotide Content Measurements

To quantify RNA/AuNP molar ratio we reacted SNAs (containing SH-OMIs-Alexa647) with 2-mercaptoethanol (20 mM final concentration) while shaking at room temperature for 5 hours. After centrifugation at 17,000 RCF (4° C.) for 30 minutes, supernatants with detached SH-OMIs-Alexa-647 were collected, transferred to 96-well Nunc-Optical Bottom Plates (Rochester, N.Y., USA) and analyzed for fluorescence measurement at 650/665 excitation and emission spectra using the Varioskan Flash Spectral Scan Multimode Reader from Thermo Fisher Scientific. The concentration was determined from a standard curve elaborated with known concentrations and fluorescence intensities of SH-OMIs-Alexa647. The molar concentrations were expressed as [RNA]/[AuNP] rations.

SNAs' In Vitro Toxicity Assay

In vitro toxicity was measured using the Alamar blue dye from Invitrogen (Carlsbad, Calif., USA) following manufacturer's instructions. Here, U87 cells (6×104 cells/ml) were seeded into 96-well cell culture plates (Eppendorf, Hamburg, Germany) and incubated for 24 hours at normal cell conditions. The next day, cells were treated with different concentrations (10 nM, 5 nM, 2.5 nM, 1.25 nM, 0.625 nM, and 0.313 nM final concentrations) of SNAs (which is attached with negative control OMI (NC-OMI). Seventy-two hours after incubation at 37° C., cell viability was determined by adding 95 μL of Alamar blue dye. Three hours later, OD values were measured in a BioRad X Mark Microplate Spectrophotometer (Hercules, Calif., USA). Cell viability was expressed in percentage using the OD of non-treated (NT) cells as 100% of viability.

Preparation of Peptide-Decorated Gold-Liposome Nanoparticles

A diagram showing the liposome preparation method used in this study is shown at FIG. 3, section B. For targeted liposomes, DSPE-PEG-2000-Maleimide micelles (previously resuspended in PBS 1×) were reacted with either ApoE (containing one cysteine at the endo of the chain) or RVG peptides (containing one cysteine at the middle of the chain) at a 1:2 molar ratio (DSPE-PEGMal:Peptide) for 72 hours at 4° C. Conjugation efficiency was monitored by calculating the amount of free maleimide or thiol groups with the Maleimide Quantification Assay Kit, and Measure IT Thiol Assay Kit, respectively. DSPE-PEG-Mal-Peptides conjugates were purified with the Pur-ALyzer Maxi 50000 Dialysis Kit (Sigma) and recovered in ddH2O.

OMIs-containing liposomes (without AuNP) were prepared as previously described by Reyes, J., et al. We mixed the OMIs with DOPC (DOPC/RNA in a 10:1 w/w ratio), Cholesterol (DOPC/Cholesterol in a 4:1 w/w ratio), and DSPE-PEG-2000 (5-10% of DOPC) and an excess of tert-butanol. The mixture was lyophilized and stored at −20 C until used. ApoE or RVG-peptide decorated liposomes were prepared by mixing DOPC and Cholesterol followed by the addition of DSPE-PEG-2000 (5-10% DOPC) and DSPE-PEG-Mal-Peptides (20% mol/mol of DOPC). The mixture was diluted with tert-butanol, lyophilized and resuspended in PBS 1×. SNAs-containing liposomes were prepared by mixing the liposomes with the previously prepared SNAs (diluted in PBS 1×).

Encapsulation Efficiency

OMIs containing liposomes were dialyzed with Pur-A-Lyzer™ Maxi 50,000 Dialysis tubes. Water (ddH2O) was changed every 30 minutes for a total period of 8 hours. After this period, 2% of Triton X-100 was added to the dialysis tubes and the amount of OMIs was quantify with a Qubit microRNA Assay Kit in a Qubit 3.0 Fluorometer (Invitrogen, Carlsbad, Calif., USA), according to manufacturer's specifications. RFU values and OMI concentrations were determined by using a standard curve prepared with known concentrations of OMIs and corrected with 2.0% of Triton-X39. The amount of encapsulated OMIs (encapsulation efficiency) was calculated as the percentage amount of OMI inside the membrane divided by the amount of OMI prior to dialysis.

To determine the encapsulation efficiency of SNA-Liposomes, SNA-Liposomes-ApoE, and SNA-Liposomes-RVG, samples were transferred to a Nanosep 300K filter columns (Palls Corporation; NY, USA) and centrifuged at 7500 RPM for 10 minutes. The first filtrate (non-encapsulated SNA) was collected and columns treated with 2% of Triton X-100 followed by an additional centrifugation at 7500 RPM for 10 minutes. AuNP concentrations in the first and second centrifugation were calculated as described above.

Nanoparticle Size and Zeta Potential Measurements

The nanoparticle's hydrodynamic diameter, charge and polydispersity were measured by Dynamic Light Scattering in a Mobius instrument (Wyatt Technologies, Santa Barbara, Ca, USA). Each sample that was analyzed had the same concentration of OMIs (12.5 μg/mL) dispersed in 1×PBS (Mg2+ and Ca2+-free) at room temperature.

Nanoparticle Uptake into GBM Cells Assessment

Internalization efficiency of nanoparticles into U87 GBM cells (3.5×104 cells/mL) were plated into Lab-Tek Chamber Slides (Thermo-Fisher) and incubated overnight at 37° C., 5% CO2 with humid atmosphere. The next day, the cells were treated with each nanoparticle preparation (liposomes, liposomes-ApoE, Liposomes-RVG, SNAs, SNAliposome, SNA-liposome-ApoE and SNA-liposome-RVG) and dissolved in Opti-MEM (Gibco, MD, USA). For this experiment, the OMI used to prepare each nanoparticle was labeled with Alexa-Fluor 647. An untreated control and a Lipofectamine RNAimax (OMM:Lipofectamine ratio of 1:1 v/v) (Invitrogen) control (positive control) were included. Cells were incubated with each treatment for 6 hours at normal cell cultured conditions. The media was removed, cells were washed with 1×PBS, fixed with ethanol 100%, stained with DAPI (1:5000), and mounted with Permafluor Mountant (Thermo Scientific). Slides were observed under a Nikon Eclipse E400 fluorescent microscope and pictures were taken with Nikon DS-Qi2 Camera. The amount of fluorescence inside the cells was quantified with NIS-Element Microscope Imaging Software. Mean fluorescent intensities were quantified by normalizing Alexa-Fluor 647 fluorescent intensities with DAPI fluorescent intensities.

In Vitro miR-92b Downregulation

U87 cells (3.5×104 cells/mL, 2 mL) were seeded into 6-well plates (Eppendorf) and incubated overnight at normal cultured conditions. The next day, cells were treated with each nanoparticle formulation containing either NC-OMM or antimiR-92b (92b-OMI). An untreated cell group and a Lipofectamine transfection reagent group were also included. Each nanoparticle formulation was dissolved in Opti-MEM cultured media and added to the cells. Seven-hours later, Opti-MEM was replaced by DME/F12 and cells were incubated overnight. The next day, cells were detached with trypsin (0.25%), collected, washed with PBS 1× and pellets of cells were stored at −80° C. until used.

RNA Isolation, cDNA Synthesis and Real-Time PCR

Total RNA (including miRNAs) was isolated with the Mirvana miRNA Isolation Kit (Invitrogen) as per the manufacturer instructions. Ten nanograms (ng) of RNA were used for cDNA synthesis with the TaqMan MicroRNA Reverse Transcription Kit (Applied Biosystems) in an Applied Biosystems Veriti 96-well Thermal Cycler (16° C. for 30 minutes, 42° C. for 30 minutes, 85° C. for 5 minutes, and 4° C. for 15 minutes). One μL of cDNA was added to TaqMan Universal Master Mix II, with UNGs (Applied Biosystems) and primers for miR-92b or U48 (internal control). PCR was performed in a StepOnePlus Real-Time PCR System (Applied Biosystems) and data was processed with the StepOne V2.3 Analysis Software. Relative expression of miR-92b was calculated by the ΔΔCt method using the U48 samples as the internal control.

Tumor Implantation and Nanoparticle Administration

Animals were handled for experiments according to the Institutional Animal Care and Use Committee (IACUC) from the University of Puerto Rico, Medical Sciences Campus. GL261 cells were implanted unto the right striatum of 12 weeks-old C57BL/6 mice from Taconic Biosciences (Rensselaer, N.Y., USA) following previously described methods. In brief, the animals or mammals were anesthetized with intraperitoneal injections of ketamine cocktail. We made a middle scalp incision and localized them at a Digital Just for Mouse Stereotactic Equipment from Stoelting (Wood Dale, Ill., USA). A burr hole-2 mm lateral and 2 mm anterior from the bregma anatomical site-was made on the scalp of each animal using a Quintessential Stereotaxic Injector and a 10 μL (0.48 mm).

Using a Hamilton Syringe (Stoelting), we injected 3×105 cells (1.5×105 cells/μL in PBS 1×) at a 0.2 μL/min flow rate-2 mm ventral from drilled scalp. Each nanoparticle formulation (containing 10 μg of OMIs) was administered (i.v.) two weeks after cells implantation. The following groups of mice (N=4 per condition) were included in this set of experiments: (a) saline solution, (b) Liposome OMIs, (c) Liposome-ApoE OMIs, (d) Liposome-RVG OMIs, (e) SNA-Liposome OMIs, (f) SNALiposome-ApoE, and (g) SNA-Liposome-RVG. The Liposomes used in this set of experiments contained 1% (of DOPC, w/w) of the fluorescent dye 3,3′Diotadecyloxacorbocyanine perchlorate (DiO, Sigma).

Immunofluorescence and Nanoparticle Accumulation Analysis

Six hours post-treatment, mice were anesthetized and transcardially perfused with PBS 1×, followed by 4% paraformaldehyde (PFA). The whole brains were incubated with PFA for 24 hours, then changed to PBS 1× for 24 hours, stored in ethanol 70% and embedded in paraffin. Brain slides (diameter) were immunostained with an antibody against the glial fibrillary acidic protein (GFAP-Green) to localize astrocytes, reactive gliosis and GBM foci. We also counterstained with 4′,6-diamidino-2-phenylindole (DAPI-blue) for nuclear identification. Briefly, brain slides were subjected to deparaffinization, followed by antigen site retrieval, quenching of endogenous peroxidase, blocking of nonspecific epitopes and serial incubation with a rabbit anti-GFAP polyclonal antibody (1:700, Abcam, Carlsbad, Calif., USA) and goat anti-rabbit monoclonal antibody (1:200, AlexaFluor 488, Abcam, Carlsbad, Calif., USA). Nuclear staining was performed with DAPI (1:15,000) following standard immunofluorescence staining protocols 1,2,3. Tissue slices was observed under a Nikon Eclipse Ts2R microscope. Images were taken (40×) with a Nikon DS-Qi2 camera and subsequently analyzed with the NIS-Element Microscope Software.

Endosomal Escape Analysis of SNA-Liposome-ApoE Nanoparticles

U87 cells (3.5×104 cells/mL) were seeded into Lab-Tek Chamber Slides (Thermo-Fisher) and maintained under standard cell conditions. The next day, cells were treated with SNA-Liposome-ApoE nanoparticles containing Alexa-Fluor 647 labeled OMIs at a final concentration of 100 nM (OMIs) in Opti-MEM media. Cells were incubated for 2, 6, and 24 hours, followed by ethanol fixation. Then, lysosomes and late endosomes were marked with the rabbit polyclonal antibody against Lamp-1 (1:200, Abcam) followed by the goat monoclonal anti-rabbit IgG (secondary antibody) labeled with Alexa-Fluor 488 (1:200, Abcam). Cells were then counterstained with DAPI (1:5000), and slides were mounted. U87 cells were observed under a Nikon Eclipse E400 fluorescent microscope, and images were acquired at 60× magnification with the Nikon DS-Qi2 camera. The cell nucleus (DAPI), lysosomes/late endosomes (LAMP-1), and OMIs in the SNA-Liposome-ApoE nanoparticles (Alexa-Fluor 647) were identified in blue, green, and red colors, respectively.

Cell Viability of miR-92b-Targeted SNA-Liposome-ApoE

The Alamar blue dye assay (Thermo Fisher) was used to measure the cell viability of U87 cells after treatment with SNA-Liposome-ApoE targeting miR-92b. U87 cells (5×104 cells/mL) were plated into 96-well plates and incubated under normal cell conditions. The next day, cells were treated with the SNA-Liposome-ApoE nanoparticle carrying 50 nM or 25 nM of OMIs (NC-OMIs and miR92b-OMIs) in Opti-MEM media and incubated under normal cell conditions. Seventy-two hours post-treatment, the media was replaced with 95 μL of Alamar blue. Three hours later, OD values were measured, and the cell viability % analyzed as (technical replicates) relative to the non-treated control (cell viability of 100%).

Serum OMIs Stability and SNA-Liposome-ApoE Shelf-Life

For serum stability, naked-OMIs and SNA-Liposome-ApoE nanoparticles were incubated in 30% FBS at 37° C. for 0, 24, 48, and 72 hours. Aliquots (containing 2 μg of RNA) were collected at each time point and separated in a 2% agarose gel electrophoresis. RNA bands were imaged using the ChemiDoc MP Imaging System (Bio-Rad). The shelf-life of SNA-Liposome-ApoE was determined by evaluating the conservation of the nanoparticle's hydrodynamic diameter and PDI of SNA-Liposome-ApoE at room temperature (25° C.) for 0, 4, 8, and 24 hours. At each time point, the size (diameter), zeta potential, and PDI were measured by DLS with a Mobius instrument (Wyatt Technologies, Santa Barbara, Calif., USA).

Statistical Analysis

Each experiment was performed at least in triplicates. We used GraphPad Prism 5 software (GraphPad Software, Inc., La Jolla, Calif., USA) for graph construction and statistical analysis. Data was analyzed with Student's t-test for comparing two groups and one-way ANOVA for multiple group comparisons (Tukey's Post Test). Results of *p<0.05, **p<0.01, and ***p<0.001 were considered significant.

Results

Conjugation of OMIs to AuNPs

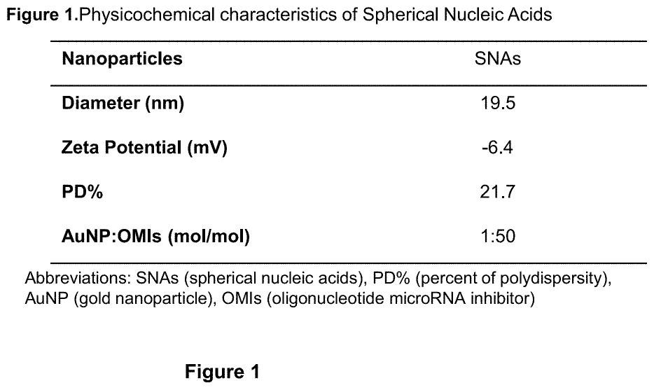

The addition of a sulfhydryl (thiol) group on the 3′-end of OMIs enables RNA-AuNP functionalization through a covalent thiol-gold bond. According to Jiuxing Li, et. al., PEG and Tween-20 can synergistically stabilize AuNPs, preventing in this way AuNP aggregation. By using this approach, conjugation of OMIs to AuNP was achieved in 1.5 hours. Table 1 shows the size, the charge and the polydispersity of the OMIs-AuNPs (SNAs). To determine the number of OMIs per AuNP, we broke the gold-thiol bond with the reducing agent 2-ME. OMIs were separated from AuNPs by centrifugation. Results showed that around that 1 mole of AuNPs contains 50 moles of OMIs (see FIG. 1).

SNAs are Nontoxic to GBM Cells

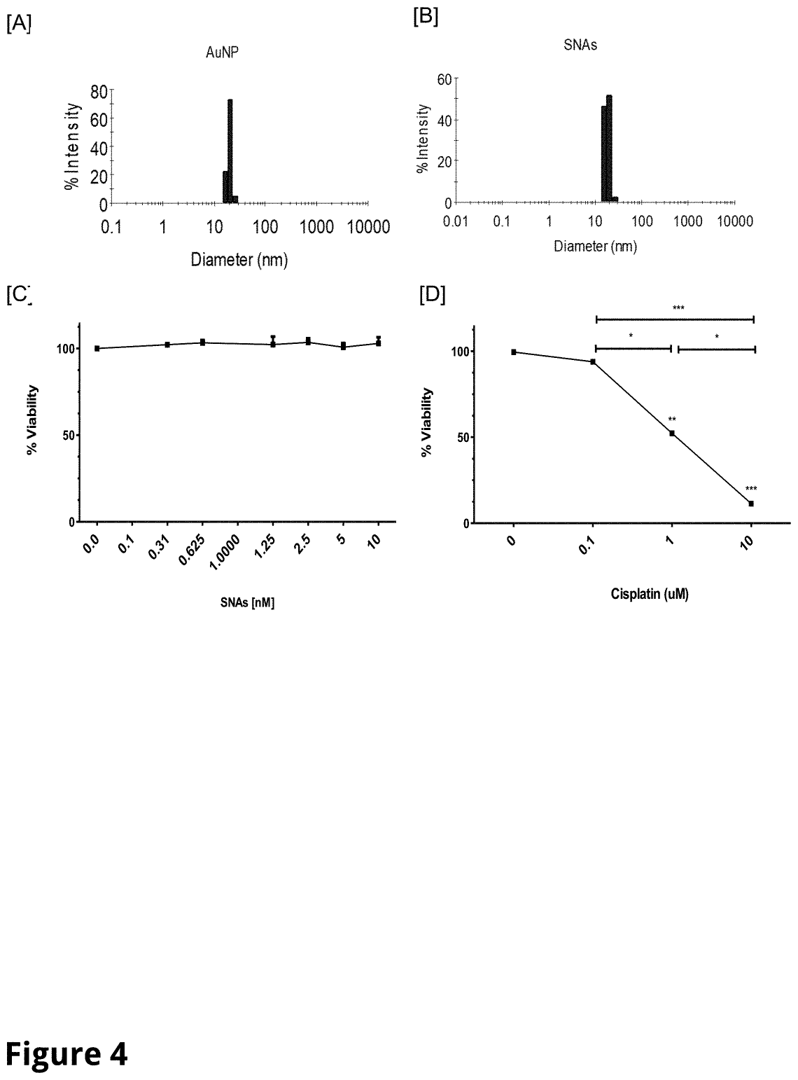

Although AuNPs are considered inert and non-toxic to the cells, their functionalization can result in toxic particles. Incubation of U87 cells with different concentrations of OMIs-AuNPs (0.3 nM to 10 nM OMIs or 15 nM to 500 nM OMIs, respectively) for 72 hours was not toxic to the cells at any of the concentrations tested (FIG. 4, section C). As a positive control of this experiment, we incubated U87 cells with cisplatin (0.1 μM to 10 μM) for 72 hours. Cisplatin is a chemotherapeutic agent commonly used against solid tumors. The highest cisplatin concentration reduced cell viability by more than 90% (FIG. 4, section D).

DSPE-PEG-Mal-Peptide Conjugation Efficiency

Brain endothelium expresses high levels of lipoprotein receptors which can interact with ApoE peptide and enable crossing of the BBB. On the other hand, RVG peptide sequence is derived from a neurotropic virus, with specificity for the nicotinic acetylcholine receptors (nAchR), present in brain cells and brain endothelial cells. GBM cells also express high levels of both LDL receptors and nAchR. Thus, we conjugated ApoE or RVG peptides to DSPE-PEG-Mal to prepare targeted liposomes. The amount of ApoE or RVG conjugated to DSPE-PEG-Mal was monitored with the Maleimide Quantification Assay Kit and Measure IT Quantification Kit which detect free maleimide and free thiol groups, respectively. By using these procedures, we obtained more than 90% conjugation efficiency of each peptide with DSPE-PEG-Mal.

Liposome Preparation and Characterization

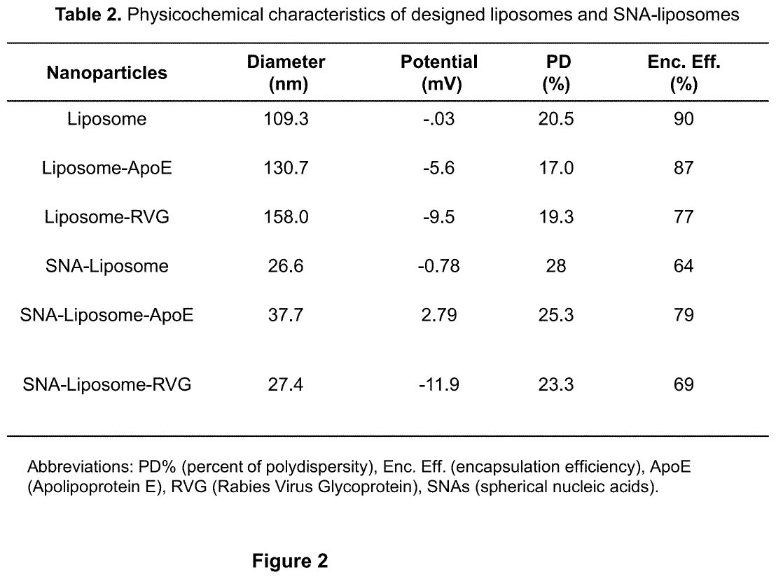

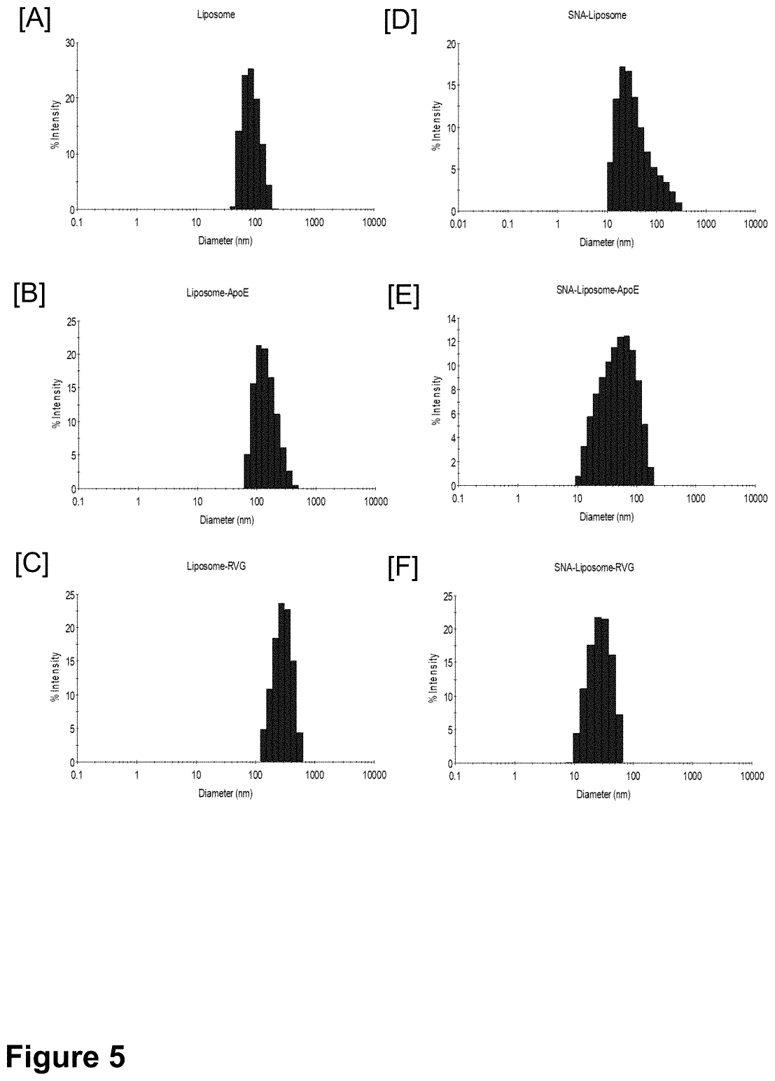

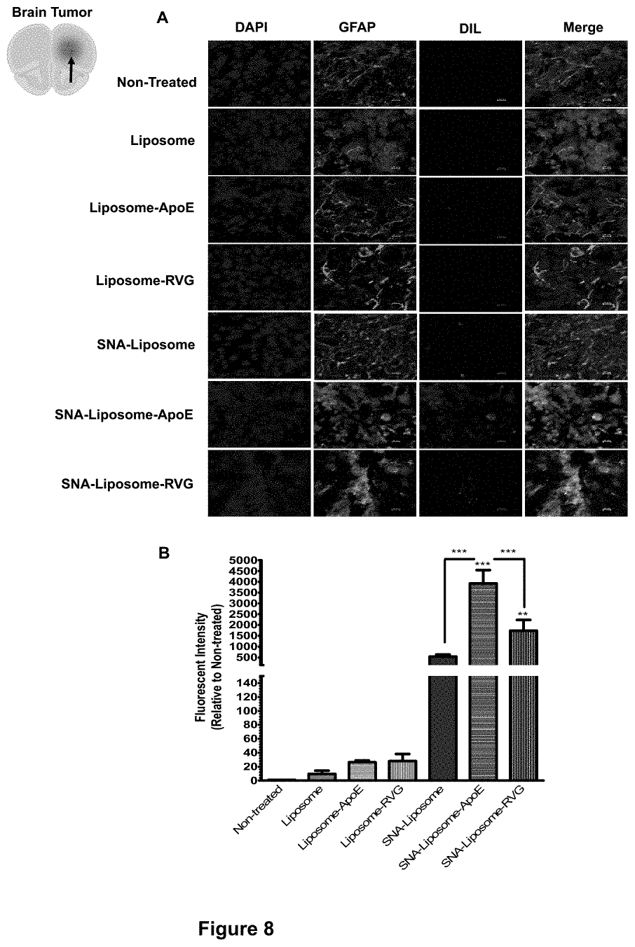

To synthesize brain-targeted nanoparticles, we first conjugated ApoE or RVG peptides to DSPE-PEG-Maleimide micelles, as shown in FIG. 3, section B. Liposomes were prepared by mixing OMIs, DOPC, Cholesterol, DSPE-PEG(2000) and peptide-DSPE-PEG conjugates in the proportions described in the “Materials and Methods” section. FIG. 2 shows the diameter, charge, and PD % of each nanoparticle. FIG. 5 shows the DLS histograms for the nanoparticle size distribution. These DLS results, as shown in FIG. 2 and FIG. 5, show that SNA-Liposomes, SNA-Liposome-ApoE, and SNA-Liposome-RVG are around three times smaller than their paired OMIs-containing liposomal formulations Liposome, Liposome-ApoE, and Liposome-RVG, as shown in FIG. 2 and FIG. 5. SNA-containing liposomes ranged from 27 nm to 42 nm, compared to OMIs-containing liposomes, which ranged from 100 nm to 150 nm, as shown in FIG. 2 and FIG. 5. Curiously, the size of AuNP-PEG-Liposomes (AuNPs without OMIs) was similar to the SNA-Liposomes, as show in FIG. 8. The zeta potential of the liposomes and SNA-Liposomes were neutral (−10 mV to +10 mV). FIG. 2 shows the encapsulation efficiency of OMIs (for Liposome, Liposome-ApoE, and Liposome-RVG) and SNAs (for SNA-Liposomes, SNA-Liposome-ApoE, and SNA-Liposome-RVG). The diameter, polydispersity and encapsulation efficiency of these liposome formulations are shown in FIG. 2. In addition, all liposomes were able to encapsulate more than 70% of all OMIs, for example see FIG. 2.

SNA-Liposome Preparation and Characterization

To prepare SNA-Liposomes, lyophilized liposomes were mixed with SNAs particles (previously resuspended in PBS), followed by vortex for 5 minutes and sonication for 10 minutes. The diameters of SNAs containing liposomes ranging from 26 nm to 40 nm approximately as shown in FIG. 1.

The size of SNA-Liposomes with RVG or ApoE peptides were more than three times smaller than their respective non-targeted liposomes, see FIG. 1. All particles exhibited a polydispersity of less than 30% and around 64% to 79% of the SNAs were encapsulated inside liposomes, see FIG. 1.

In Vitro Cell Internalization Experiments

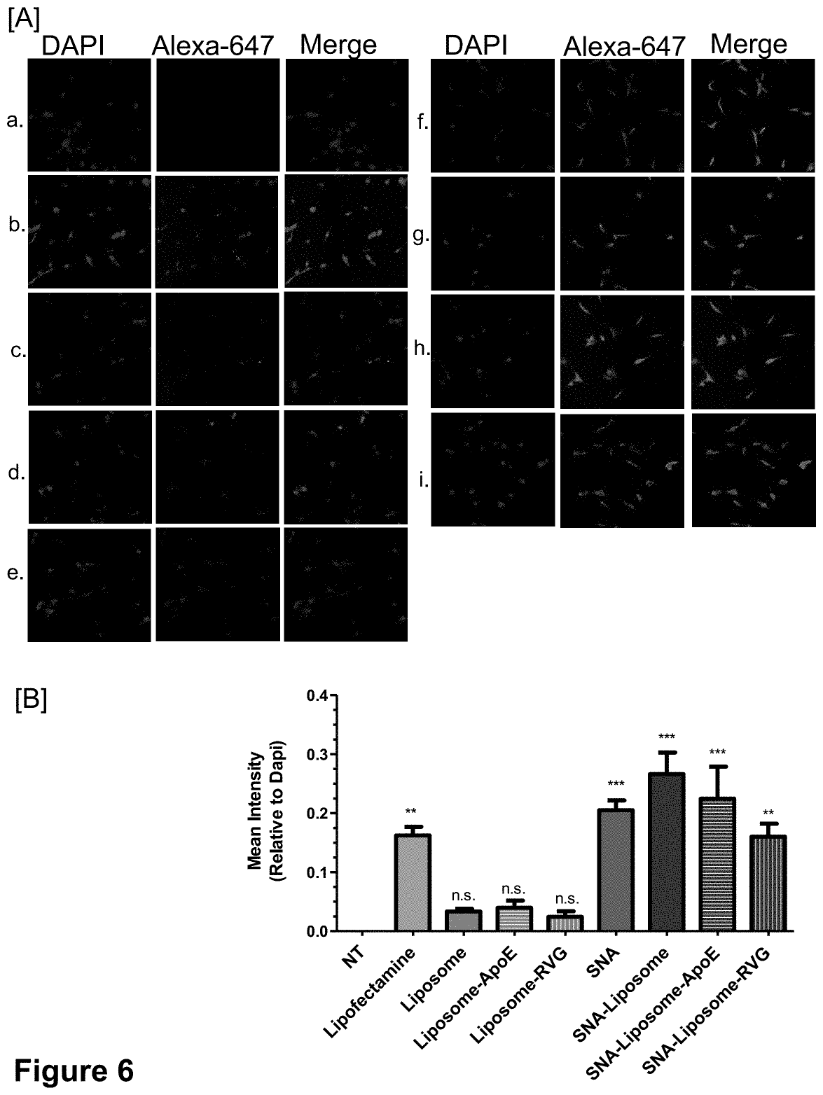

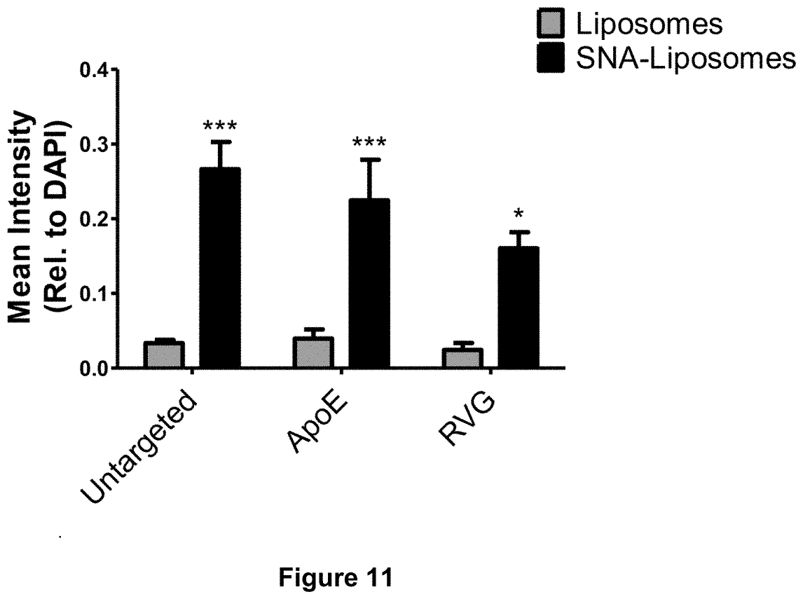

We treated U87 cells with each nanoparticle (containing 100 nM of Alexa-647 labeled OMIs) and six hours later cells were fixed and stained with DAP for fluorescent microscopy analysis. The microscopy images showed that all nanoparticles were able to internalize into GBM cells (FIG. 6). Both, labeled and unlabeled SNA-Liposomes exhibited high significant internalization rates (<0.01 and <0.001) compared with non-treated groups and all liposome formulations (<0.05). Interestingly, the internalization efficiency of SNAs (<0.001) alone was comparable with the lipofectamine positive control and SNA-liposomes. Additional statistical analysis showed that SNA-Liposomes, SNA-Liposome-ApoE, and SNA-Liposome-RVG significantly increase OMIs internalization into U87 cells when compared to their liposomal counterparts, Liposome (***P<0.001), Liposome-ApoE (***P<0.001), and Liposome-RVG (*P<0.05), respectively, as shown in FIG. 11. SNAs and all SNA-containing liposomes had similar internalization efficiencies compared with Lipofectamine-transfected OMIs, as show in FIG. 6, section B.

Downregulation of miRNA-92b

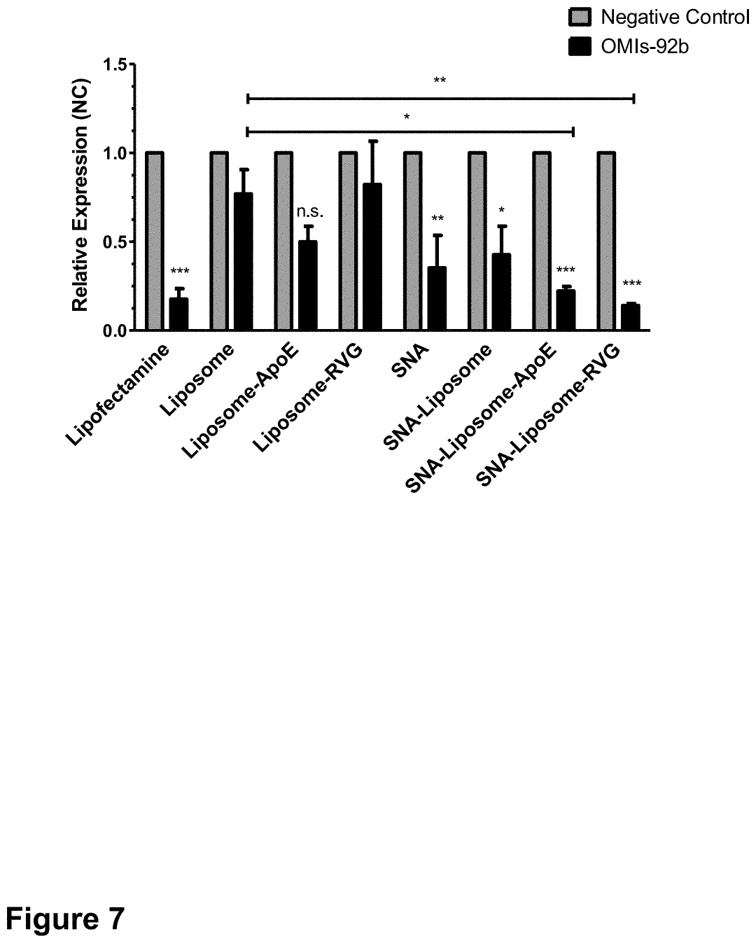

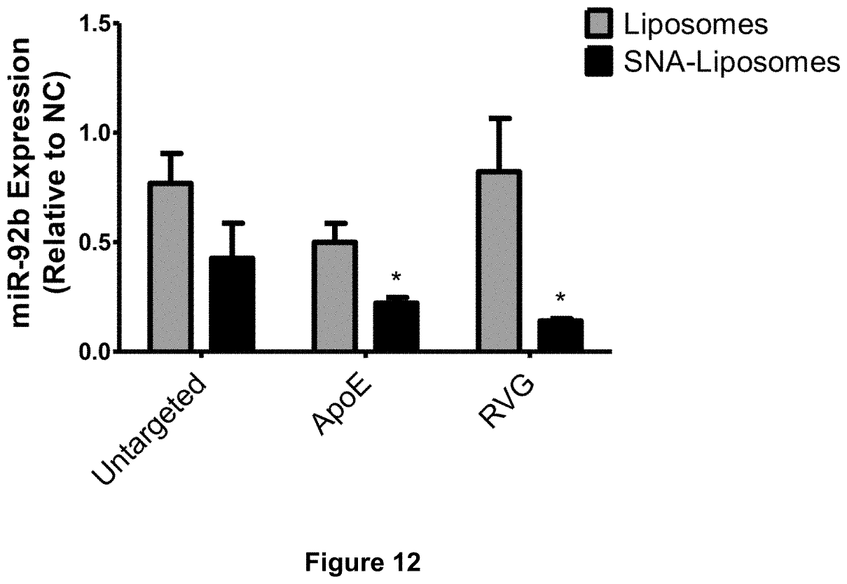

We treated U87 cells with all nanoparticles containing either miR-92 OMI or NC-OMI and 24-hours after treatment we measured the miRNA-92b levels by qPCR analysis, as shown in FIG. 7. As expected miR-92b relative expression in U87 cells was significantly reduced in the Lipofectamine assisted control (<0.001). Congruent with our previous internalization data, SNAs (<0.01), SNA-Liposome (<0.05), SNA-Liposome-ApoE (<0.001), and SNA-Liposome-RVG (<0.001) were superior inhibiting the expression of miR-92b in U87 cells compared to their negative control (NC) formulations. SNA-Liposome-ApoE (<0.05) and SNA-Liposome-RVG (<0.01) were also more effective inhibiting miR-92b in comparison to the unlabeled formulation of Liposome containing OMIs-92. Although not significant, Liposome-ApoE formulation showed a trend to decrease miR-92b expression. Remarkably, SNAs and SNA-Liposomes downregulated miR-92b at the same extension than lipofectamine RNAimax, a transfection reagent amply used to transfect RNA-OL in cultured cells, as shown in FIG. 7. Together, these results indicate that targeted nanoparticles not only efficiently internalized OMI inside cells but also effectively downregulated the targeted miRNA (miR-92b). Additional statistical analysis, as shown in FIG. 12, showed a significant decrease in the relative expression of miR-92b in U87 cells after treatment with SNA-Liposome-ApoE (*P<0.05) and SNA-Liposome-RVG (*P<0.05) compared to Liposome-ApoE and Liposome-RVG respectively. Together, these results indicate that ApoE and RVG peptides-targeted SNA-Liposome nanoparticles promoted cell internalization and reduced miR-92b levels in GBM cells.

Accumulation of Nanoparticles in Brain Tumor Tissue

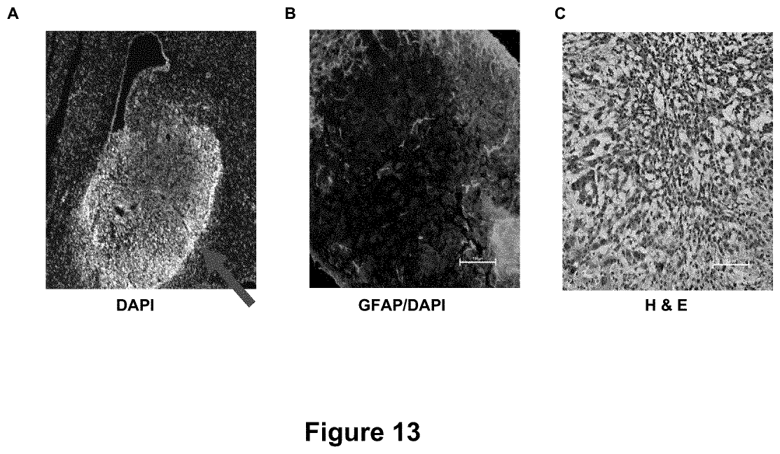

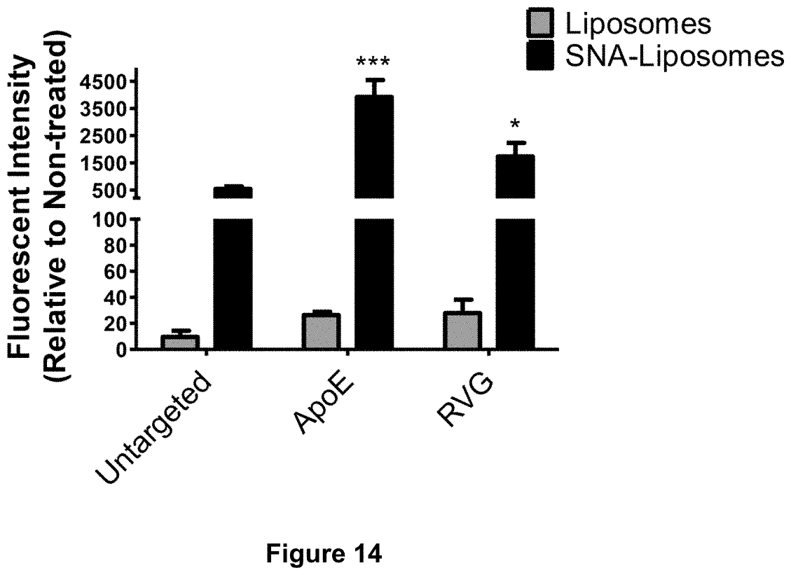

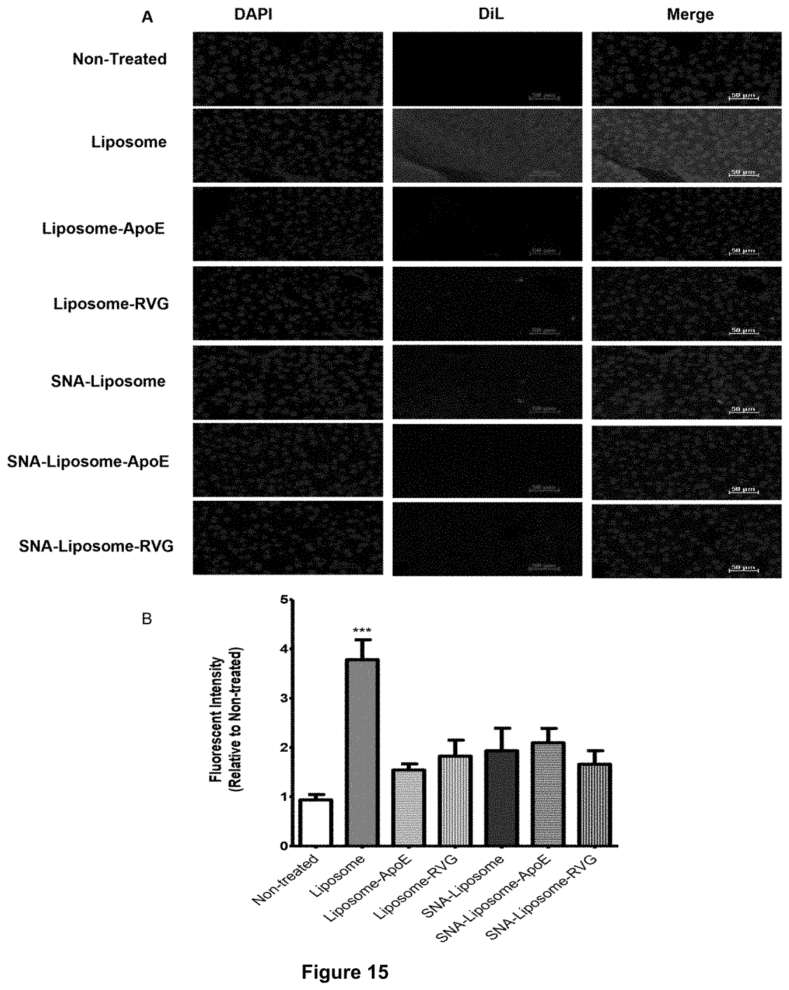

The presence of brain tumors was confirmed by H&E, DAPI staining (to identify enlarged nuclei), and GFAP immunofluorescence staining (to localize astrocytoma tumor foci), as shown in FIG. 13. Two weeks after tumor implantation, we injected mice intravenously (iv) with PBS (control non-treated group) or with each nanoparticle formulation (Liposome, Liposome-ApoE, Liposome-RVG, SNA-Liposome, SNA-Liposome-ApoE, and SNA-Liposome-RVG). Liposomes were stained with DiL fluorescent lipid dye (see methods). Six hours later, mice were perfused and fixed to process the brain tissues for immunofluorescence staining and nanoparticle localization in brain tumors. To localize brain tumors and DiL (red) stained nanoparticles, we immunostained brain slices against GFAP (green) and counterstained with DAPI (blue) (FIG. 8, section A). Our microscopy images showed prominent tumor accumulation of the following nanoparticles: SNALiposome, SNA-Liposome-ApoE, and SNA-Liposome-RVG (FIG. 8, section A). Of all, SNA-Liposome-ApoE and SNA-Liposome-RVG colocalized (internalize) with the tumor tissue cells (FIG. 8, section A, merge images). FIG. 8, section B shows the analysis for red fluorescent intensities (quantified with the NIS-Element Microscope Imaging Software) localized in brain tumors after nanoparticle administration. We observed a significant increase in fluorescent intensities for SNA-Liposome-ApoE (***P<0.001) and SNA-Liposome-RVG (**P<0.01) compared with the non-treated group, as shown in FIG. 8, section B. Notably, the SNA-Liposome-ApoE nanoparticles showed a significant accumulation in the brain tumor area compared to SNA-Liposomes (***P<0.001) and SNA-Liposome-RVG (***P<0.001). We also analyzed the fluorescent intensities of SNA-Liposome, SNA-Liposome-ApoE, and SNA-Liposome-RVG compared to their OMIs containing liposomal counterparts (Liposome, Liposome-ApoE, and Liposome-RVG, respectively). This analysis showed a significant increase in the fluorescent intensities of SNA-Liposome-ApoE (***P<0.001) and SNA-Liposome-RVG (*P<0.05), as shown in FIG. 14. Also, fluorescence microscopy images taken from brain tumor-adjacent areas showed no DiL-associated fluorescence in any of the formulations tested, as shown in FIG. 13. Interestingly, the Liposome formulation showed higher accumulation in the mice liver tissue compared with the other nanoparticles (***P<0.001), as shown in FIG. 15. Altogether, this data suggests that systemic injections of SNA-Liposome-ApoE increased the delivery of OMIs into brain tumors.

SNA-Liposome-ApoE can Escape Lysosome/Endosomes

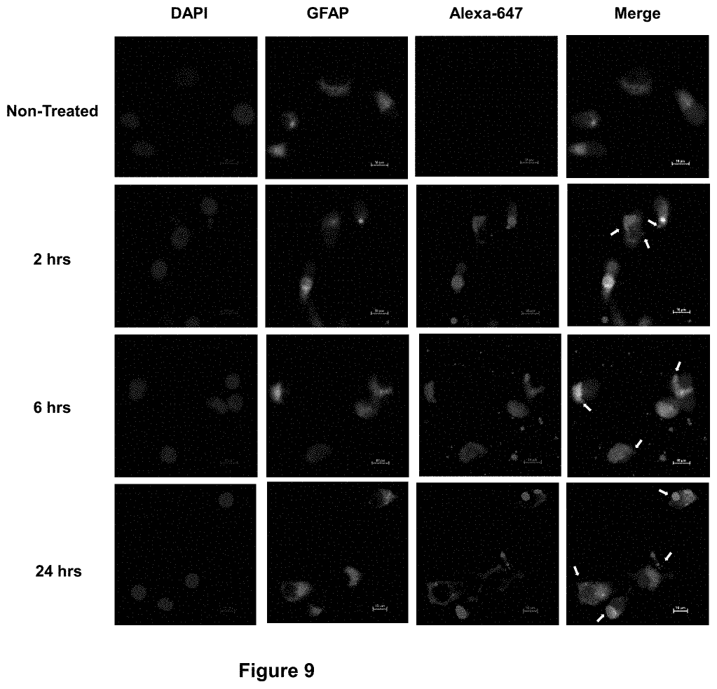

As the SNA-Liposome-ApoE nanoparticle showed the highest accumulation in U87 cells and GBM tumors, we studied the lysosomal/late endosomal fate of these nanoparticles. We treated U87 cells with SNA-Liposome-ApoE nanoparticles for 2, 6, and 24 hours followed by cell fixation and immunostaining against the lysosome/late endosome marker, Lamp-1.29,72,73,81 Fluorescent images (60× magnification) of non-treated and SNA-Liposome-ApoE treated cells showed the internalization of Alexa-Fluor 647 labeled OMIs (red) in all of the three-time points tested, as shown in FIG. 9. We detected the greatest colocalization (yellow) between SNA-Liposome-ApoE nanoparticles (red) and lysosome/late endosomes (green) at 2 hours post-treatment, although colocalization is seen at all time points, as shown in FIG. 9. We identified higher non-colocalized cell regions (white arrows) as lysosome/late endosome escape at 6 hours and 24 hours. Our results suggest that SNA-Liposome-ApoE can notably escape lysosome/late endosomes from U87 cells after 6 hours and 24 hours of nanoparticle incubation, as shown in FIG. 9.

MiR-92b-Targeted SNA-Liposome-ApoE Nanoparticles Reduced Cell Viability

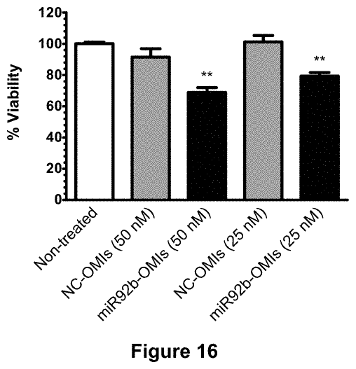

As SNA-Liposome-ApoE was accumulated in tumor tissue, it reduced miR-92b levels, and was able to escape from endosomes, we studied the capacity of miR-92b-targeted SNA-Liposome-ApoE to reduce cell viability in U87 cells. Our results showed that miR92b-OMIs in SNA-Liposome-ApoE significantly reduced cell viability at 50 nM (23%, ***P<0.001) and 25 nM (21%, **P<0.01) compared with NC-OMIs SNA-Liposome-ApoE, as shown in FIG. 16.

Serum OMIs Stability and SNA-Liposome-APOE Shelf-Life Preservation

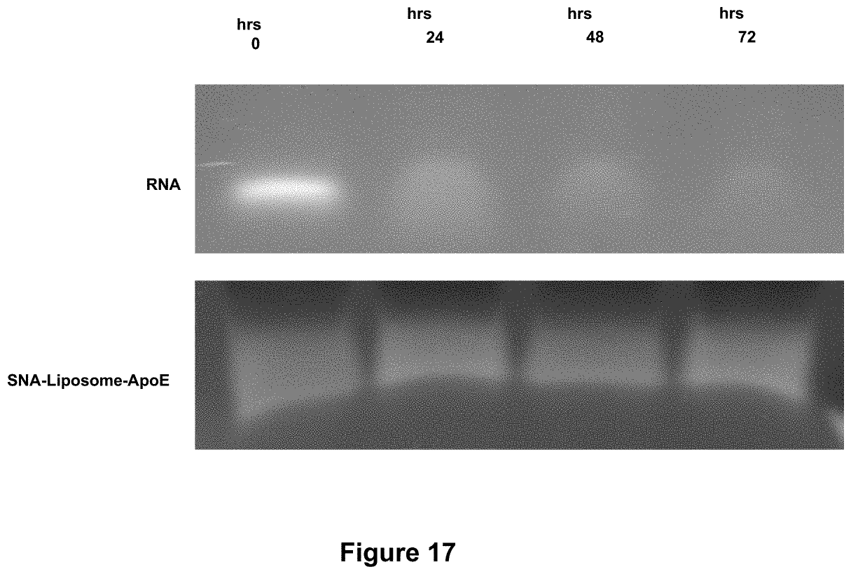

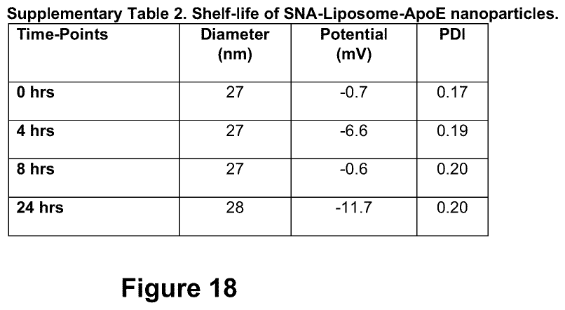

Finally, we studied the capacity of the SNA-Liposome-APOE formulation to protect OMIs from degradation by nucleases. To in vitro imitate plasma conditions, we incubated SNA-Liposome-APoE and naked OMIs in 30% FBS at 37° C. for 0, 24, 48, and 72 hours. The gel electrophoresis shown in FIG. 17 showed that naked OMIs were degraded at 24-hr of FBS incubation and were almost undetectable after 72-hr of FBS incubation. Opposite, incubation of SNA-Liposome-ApoE protected OMIs from degradation at all time-points tested, as shown in FIG. 17. When we measured the shelf life of the SNA-Liposome-ApoE, we observed that the size, charge, and PDI index were preserved at RT for 0, 4, 8, and 24 hours, as shown in FIG. 18. Altogether, these studies indicate that SNA-Liposome-ApoE can protect OMI molecules from FBS degradation and maintains liposomal physical properties at RT for at least 24 hours.

The present disclosure is related to a synthesized brain targeted gold-liposomal nanoparticle that can effectively deliver RNAi molecules to GBM tumor cells. Specifically, SNA-Liposome-ApoE increased OMIs internalization into GBM human cells, decreased miR-92b expression, reduced GBM cell viability, and increased nanoparticles' accumulation into intracranial GBM mouse tumors. GBM is among the deadliest types of cancers and the second most common type of primary brain tumor (National Cancer Institute). Also, GBM's overall survival of patients (15 months) has not improved over the last two decades. Lacking effective therapies is attributed to a myriad of reasons, including the GBM's recurrent and resistive nature and the inability of drugs to cross the BBB—which precludes more than 98% of therapies from reaching the brain. Therefore, the targeted gold-liposomes synthesized in this study promotes the advancement of RNAi-based therapies against GBM and other CNS disorders.

SNAs are spherical nucleic acids formed by a gold-thiol bond between SH-oligonucleotides and the AuNP core. Oligonucleotide AuNP interactions enable nucleic acids to be densely packed and radially orientated. The densely packed orientation of nucleic acids increases the affinity constant between complementary oligonucleotide sequences, and in turn, protects them from nuclease degradation. The unique 3D structure of SNAs allows them to effectively enter cells by Class A Scavenger Receptors via a caveola dependent pathway. Studies by Melamed, et al. demonstrated that this 3D orientation led to the increased SNA's cellular uptake in comparison to nucleic acid polyplexes. Additional work by Jensen, et al. showed that SNAs are capable of internalizing over 50 different types of cells (including U87 cells) and even crossing the BBB. A clinical trial for GBM treatment using SNAs (UN-0129) carrying siRNAs against Bcl2L12 (NCT03020017) is currently underway. Despite promising results, studies reported by Wilhelm, et al. show that less than 1% of SNAs and other nanoparticles reach tumor tissues while highly accumulating in the liver and spleen.

The lack of knowledge about gold toxicity, as well as in aggregation and excretion in vivo, increases concerns regarding continuous treatment and long-term consequences of SNA administration in patients. Therefore, tissue-specific modifications are essential to improve the delivery of SNAs to GBM tumors rather than other extraneural tissues. To enhance the specificity of GBM cells in the brain, we encapsulated SNAs inside RVG, and ApoE peptide conjugated liposomes, the liposome formulation we used (DOPC, cholesterol, DSPE-PEG-2000) to encapsulate SNAs and OMIs. These liposomes showed negligible toxicity both in vitro and in vivo, and no detectable immune responses. Also, these liposomes efficiently delivered c-MYC-targeted siRNA in a xenograft mouse model of ovarian cancer and miR-143 targeted OMIs in a subcutaneous GBM mouse model. The dual nanoparticles we designed in this study, composed of OMIs-AuNPs (SNAs) and brain targeted liposomes, have two desirable characteristics: (i) highly oriented oligonucleotides that increase inhibition efficiency and (ii) tumor specificity to improve accumulation in brain tumors. These characteristics are essential to decrease peripheral nanoparticle degradation while improving their tissue specificity, as shown by the SNA-Liposome-ApoE and SNA-Liposome-RVG nanoparticles we prepared.

Unexpectedly, our SNA-Liposome nanoparticles were around 30-50 nm in diameter when their paired liposomes (containing OMIs) were twice to three times bigger (diameters between 100-150 nm). The diameter of PEG-AuNP liposomes (without OMIs) resembles SNA-Liposomes (with OMIs), suggesting that intermolecular forces between AuNPs and liposomal components may contribute to the observed size reduction. As an approximation and assuming nanoparticles as spheres, we can estimate the volume of an SNA (10 nm radium) to be 4,190 nm³. The mean size of the SNA-liposome was 20 nm (radium). Its volume is approximately 33,520 nm³, meaning that the number of entrapped SNA into liposomes could be less than eight SNAs per liposome. Previous studies have shown that AuNPs can interact with head-groups from the lipid molecules of liposomes and thus alter their physicochemical properties. Charged interactions between AuNPs and lipidic polar groups may occur, resulting in the reduction of nanoparticle size, as observed in our study. This effect on size reduction could increase their chances of crossing the BBB. Early reports showed that nanoparticles with diameters around 50 nm could accumulate with higher efficiency inside cells, escape phagocytic uptake, and deposit in tumor tissue due to the EPR effect.

The nanoparticles of the present disclosure have neutral to slight negative charges (+10 mV to −10 mV). Evidence indicates that neutral nanoparticles are ideal for drug delivery due to their capacity to interact with cell membranes and to reduce immune response activation. Even though nanoparticles with positive charge interact easier with cell membranes, they can increase macrophage entrapment and immune response and the production of reactive oxygen species (ROS). On the other hand, negatively charged nanoparticles cannot interact with cell membranes, decreasing in this way their internalization efficacy. Adding DSPE-PEG-2000 to our nanoparticle formulations decreases their probability of being entrapped by the mononuclear phagocyte system (MPS). However, the physicochemical properties could change once nanoparticles are administered systemically due to the formation of protein corona (modifications in the surface of nanoparticles), which could alter not only the charge but other nanoparticle physicochemical properties as well.

In accordance with the principles of the present disclosure a brain targeted nanoparticle that could efficiently deliver RNAi molecules into GBM tumors is presented. The selected peptides were ApoE and RVG because of their promising results delivering biomolecules to the brain parenchyma. ApoE and RVG peptide labeled nanoparticles have enhanced the delivery of antifungal treatments, siRNAs, and proteins across the BBB and into the CNS. ApoE is a small peptide that targets LDL receptors, commonly present in brain endothelium and GBM cells. On the other hand, RVG is a neurotropic virus peptide that binds to the nicotinic acetylcholine receptors and facilitates nanoparticle delivery to the brain and brain tumors. When evaluating nanoparticles capacity to internalize GBM cells and inhibit the expression of miR-92b in vitro, we observed that SNAs, SNA-Liposomes, SNA-Liposome-ApoE, and SNA-Liposome-RVG had similar delivery efficiencies than the lipofectamine used as a positive control. Our in vivo results confirmed our hypothesis that ApoE and RVG peptides would enable RNAi systemic delivery into brain tumor cells. Specifically, SNA-Liposome-ApoE significantly improved tumor accumulation compared to each of the other nanoformulations created. Also, SNA-Liposome-ApoE was able to achieve endosomal escape from U87 GBM cells. Endosomal escape serves as an indicator of OMIs release into the cytoplasm, hence demonstrating the nanoparticle's effective delivery. Furthermore, the OMIs contained in SNA-Liposome-ApoE were protected from FBS degradation suggesting a higher payload of OMIs into the brain tumors when exposed to high protein and nuclease concentration present in plasma.

Therefore, the present disclosure provides a formulation for treating cancer comprising nanoparticles composed of SNAs encapsulated inside ApoE or RVG peptide-conjugated liposomes for the delivery of RNAi-based therapies and other drugs to brain tumors and other brain-related diseases.

Further, it will be apparent to those skilled in the art that various modifications and variations can be made to the method and system described in the present disclosure. Other embodiments of the method and system will be apparent to those skilled in the art upon consideration of the specification and practice of the method and system disclosed herein. It is intended that the specification and examples be considered as exemplary only, with a true scope of the disclosure being indicated by the claims.

1. A nanoliposomal formulation for treating cancer comprising nanoparticles composed of SNAs encapsulated inside a peptide-conjugated liposome for the delivery of RNAi-based therapies.

2. The nanoliposomal formulation of claim 1, wherein the SNAs comprise gold nanoparticles with oligonucleotide miRNA inhibitors.

3. The nanoliposomal formulation of claim 1, wherein the peptide-conjugated liposome is apolipoprotein E.

4. The nanoliposomal formulation of claim 1, wherein the peptide-conjugated liposome is rabies virus glycoprotein.

5. A method for treating cancer comprising a nanoliposomal formulation, wherein said nanoliposomal formulation comprises nanoparticles composed of SNAs encapsulated inside a peptide-conjugated liposome for the delivery of RNAi-based therapies.

6. The method of claim 5, wherein the SNAs comprise gold nanoparticles with oligonucleotide miRNA inhibitors.

7. The method of claim 5, wherein the peptide-conjugated liposome is apolipoprotein E.

8. The method of claim 5, wherein the peptide-conjugated liposome is rabies virus glycoprotein.
